(19) United States
(12) Patent Application Publication
(10) Pub. No.: US 2008/0269575 A1
(43) Pub. Date: Oct. 30, 2008

(54) Method and Apparatus for Monitoring Bodily Analytes

(76) Inventor: Gavriel J. Iddan, Haifa (IL)

Correspondence Address: MINTZ LEVIN COHN FERRIS GLOVSKY & POPEO;ATTN: PATENT INTAKE CUSTOMER NO. 35437, ONE FINANCIAL CENTER, BOSTON, MA, 02111, US

(21) Appl. No.: 11/884,644

(22) PCT Filed: Feb. 14, 2006
(86) PCT No.: PCT/IL06/00193

§ 371 Date: Apr. 16, 2008

Related U.S. Application Data
(60) Provisional application No. 60/653,504, filed on Feb. 17, 2005.

(51) Int. Cl. A61B 5/00

(52) U.S. Cl. 600/316; 600/310

(57) ABSTRACT

The present invention relates mainly to a method and apparatus for monitoring bodily analytes, such as glucose, by use of an analyzer (100) configured for emitting radiation oriented to impinge on a selected area of the skin (S), for collecting returned imprinted radiation exiting the skin, for processing and analysis of the imprinted radiation, and for display of analysis results. At least one retroreflector (RR, RI 1, R1 & 2) is inserted subcutaneously to the selected area (SA) for receiving radiation and returning retroreflected radiation, as returned imprinted radiation. The retroreflector (RR, R1 1, R1 & 2) has at least one level of retroreflection (RR1L, RR2L).

### TECHNICAL FIELD

The method and the device of the present invention relate in general to measurements of the concentration of bodily analytes by use of optical illumination, and more particularly to the measurement of glucose concentration in the body by use of radiation absorption spectroscopy. A minute implanted retroreflector is used to increase the sensitivity of the spectral analysis.

### BACKGROUND ART

Frequent testing of glucose levels in patients with diabetes is important for recognizing emergency situations and preventing the immediate and potentially serious consequences of very high or very low glucose levels. Monitoring also enables tighter glucose level control, which decreases the likelihood of development and worsening of diabetic complications over time. Patients with type-1 diabetes need to measure their blood sugar many times per day. The optimal frequency of blood glucose monitoring in type-2 diabetes is unknown, although it is usually less than for patients with type-1 diabetes.

Currently the standard method for self-monitoring of blood glucose requires pricking a finger to extract a drop of blood, which blood is applied to a test strip. The reaction between the blood glucose and the chemicals on the test strip is then analyzed to provide a numerical glucose reading. The need for finger pricking is the major drawback to this method, being unpleasant and sometimes difficult to perform in long-term diabetic patients. This unpleasantness leads to inadequate patient compliance with prescribed glucose testing regimens, leading in turn to poor control of the disease with ensuing complications and increased healthcare costs.

Various methods of glucose testing have been suggested, attempting to provide a sensitive but non-invasive alternative to the standard finger pricking method. The ultimate aim would be to develop a non-invasive device for continuous glucose monitoring in combination with a glucose pump to create an artificial pancreas.

Many of the novel approaches to non-invasive glucose monitoring devices utilize optical rather than electrochemical sensor technologies. In recent years, infrared (IR) spectroscopy has emerged as a prominent analytical method. The underlying principle of absorption spectroscopic techniques relies on the phenomenon whereby when a molecule is radiated with a range of frequencies (or wavelengths), only certain wavelengths of the radiation are absorbed. Since each molecule has a unique spectral “fingerprint”, the absorption spectrum of the sample can reveal its molecular composition. The concentration of a particular molecule in the sample can be deduced from the intensity of its absorption peak.

Noninvasive approaches for glucose concentration determination in blood usually have two steps. As a first step, an apparatus is used to acquire a reading from the body without obtaining a biological sample. As a second step, an algorithm converts this reading into a glucose determination. The better the readings from the biological sample, the more accurate the measurements. Therefore, enhancement of the first step, which is the data collection, or data reception step is of primordial importance.

The International Publication Number WO 03/076883 A3, to George Acosta et al., provides an overview of the state of the art. O. S. Khalil provides a summary of the prior art in “Non-Invasive Measurement Technologies” published in “Diabetics Technology & Therapeutics”, Vol. 6, No. 5, 2004, pp. 660-697.

The present invention is dedicated mainly to a method and apparatus regarding noninvasive detection of reflected radiation for the determination of the concentration of analytes, such as glucose for example.

A typical prior art instrument, or data sampler D, for glucose monitoring based on IR absorption spectroscopy is shown schematically in FIG. 1. Simply put, a source of radiation 2, such as an illumination source 2 or light source 2, directs an incoming beam of radiation, or light beam 4 toward and through the skin S, tissue T and a blood vessel BV. Most of the radiation of the light beam 4 is scattered in all directions. Some of the scattered rays 6 exit through the tissue T and the skin S, and reach a sensor 8, which receives the returned light and detects the absorption spectrum. In turn, the concentration of particular molecules is deduced from the intensity of the relevant absorption peak, according to the spectrum received by the sensor 8.

As shown in FIG. 1, only a small fraction of the illumination beam 4 ends up reaching the sensor 8, since most of the beam is scattered in various directions. The extensive scattering phenomenon both reduces the amount of energy that returns to the sensor 8 and also allows unwanted rays of light that avoided the blood vessel BV to be received by the sensor 8. Thereby, the result is a low signal-to-noise ratio and insufficient analysis sensitivity, which is perhaps why the method did not achieve success. Increasing the sensitivity of the measuring system, or data receptor D, and increasing the specific contribution of the signal containing blood-related information are some of the main challenges for the development of a non-invasive IR spectroscopy method for glucose sensing and measuring.

In U.S. Pat. No. 6,442,409, Peyman; G. A. teaches a “Reflective device 114, which is implanted in the eye 28 of a patient, is positioned within the eye 28 to reflect outwardly the beam of radiation 120. Reflective device 114 may be coupled to, attached to or formed with surface 56 of lens portion 54, embedded into lens portion 54 or attached to the haptics 56 or any combination thereof. Reflective device 114 functions in a manner similar to light emitter 14 described above, however it does not emit a new beam of radiation but rather reflects the original beam of radiation. It has been found advantageous to use a mirror for reflective device 114. This has the advantage of being biocompatible and inexpensive. Further, it is a relatively efficient device for reflecting radiant energy. The mirror may be any suitable size, however, it has been found preferable to limit the size of the mirror in length and width to from about 0.02 mm to about 5 mm for this application, making the mirror invisible to the patient in which it is implanted. It will be readily apparent to those skilled in the art, that multiple mirrors may be implanted simultaneously in any number of locations on the intraocular lens system.”.

The invention of Peyman; G. A. is not known to have gained either acceptance in professional circles or commercial success.

There is thus a need for a method permitting to implement an apparatus for deriving biological parameters from the interior of a body, such as a concentration level of an analyte, and especially glucose, that is small, low-cost, portable, and reliable.

### DISCLOSURE OF THE INVENTION

To take advantage of the benefits offered by a reflective element, there are at least two main obstacles to overcome: to find a way to subcutaneously insert the reflector by means of a simple procedure, and to provide a method permitting to precisely locate one or more beams of radiation returned by the implanted and hidden reflector.

It is proposed to subcutaneously implant a minute retroreflector so small as to be injected below the skin by use of a hypodermic needle pertaining to a dedicated insertion tool, or to anchor or sew-in the retroreflector by help of surgical thread and needle.

A retroreflector is an optical device that sends light or other radiation back where it arrived from, regardless of the angle of incidence, unlike a mirror, which does that only if the mirror is exactly perpendicular to the light beam. The use of an implanted retroreflector thus defeats the drawbacks of the prior art: the returned retroreflected radiation is oriented exactly, but in opposite, to the direction of the impinging beam of radiation.

Thereby, most of the emitted impinging energy is returned out of the skin and to a detector, also minimizing the amount of unwanted stray rays of light received by the detector. In other words, the retroreflected radiation returns in a well known direction, thus toward the source of illuminating radiation, irrespective of the angle of incidence of the impinging beam of radiation relative to the skin.

The implanted retroreflector increases and improves data collection and reception, as well as the sensitivity of the analysis, and leads to more accurate quantification of the radiation or illumination absorbing constituents. For the sake of orientation, a retroreflector selected for implantation is as small as 0.2×0.3×1 mm and may reach the dimensions of 0.5×1×6 mm.

As opposed to other non-invasive technologies, the disclosed method and device doe not require frequent calibration, such as is common with standard finger pricking tests. The present invention is useful for both, routine monitoring of developing trends, and measurement of absolute levels of glucose, and is applicable for monitoring trends and levels of other blood analytes as well.

The subcutaneous implantation of a miniscule retroreflector consisting of the injection of the reflector into tissue under the skin, or under a blood vessel, is a procedure as simple as a familiar hypodermic injection. The same holds for the sewing in of a subcutaneous retroreflector. Such a simple implantation provides the benefit of subsequent analyte concentration readings that are obtained periodically or continuously in non-invasive, painless manner, for improved patient comfort. Accurate readings are obtained in an undemanding, easy to use, and foolproof manner, requiring no special skills or training.

### SUMMARY OF THE INVENTION

It is an object of the present invention to provide a method and apparatus for monitoring bodily analytes by use of an analyzer (100) configured for emitting radiation oriented to impinge on a selected area (SA) of the skin (S), for collecting returned imprinted radiation exiting the skin, for processing and analysis of the imprinted radiation, and for display of analysis results. The method and apparatus call for inserting at least one retroreflector (RR, R1, R2) subcutaneously to the selected area (SA), for receiving radiation and returning retroreflected radiation, and for collecting retroreflected radiation exiting from the selected area as returned imprinted radiation.

It is another object of the present invention to provide a method and apparatus for

collecting imprinted radiation separately from each at least one retroreflector (RR, R1, R2), which has at least one level of retroreflection (RR1L, RR2L). Preferably, the at least one retroreflector (RR, R1, R2) has at least two levels of retroreflection (RR1L, RR2L) mutually separated away by a step distance (2d), and radiation is received and returned separately for each one of the two levels of retroreflection (RR1L, RR2L).

It is yet another object of the present invention to provide a method and apparatus wherein returned imprinted radiation is collected as a plurality of separate portions of radiation for independent processing and analysis of each separate portion, and for combination of independent processing and analysis.

It is still another object of the present invention to provide a method and apparatus wherein the at least one retroreflector (RR, R1, R2) is disposed appropriately and configured to return retroreflected-radiation as a plurality of distinct separate imprinted beams collected for either one of both separate and combined processing and analysis. Likewise, the at least one retroreflector (RR, R1, R2) is disposed in selected position and orientation to receive radiation and return imprinted radiation as separate beams, which are collected, processed, and analyzed independently and in combination.

It is one more object of the present invention to provide a method and apparatus wherein the at least one retroreflector (RR, R1, R2) is configured and disposed in selected position and orientation to receive radiation and return radiation crossing at least the skin (S), and tissue (T). Thus, the at least one retroreflector (RR, R1, R2) is configured and disposed in selected position and orientation to at least receive and return a first portion of radiation crossing the skin (S), tissue (T), and a blood vessel (BV), and to at least receive and return a second portion of radiation crossing the skin (S) and tissue (T). Furthermore, radiation may also cross a blood vessel (BV), and a muscle (M) or a portion thereof. Moreover, the received and returned radiation have at least a first portion and a second portion of radiation crossing bodily matter via a separate, respectively, first path and a second path, and the first path and the second path cross different bodily matter.

It is one further object of the present invention to provide a method and apparatus wherein the at least one retroreflector (RR, R1, R2) is not larger than 0.2 mm×0.3 mm×1 mm, or not larger than 0.5 mm×1 mm×6 mm.

It is yet a further object of the present invention to provide a method and apparatus wherein subcutaneous insertion into a selected location (TL) is provided by ejection of the at least one retroreflector (RR, R1, R2) out of a hypodermic needle (205), where the hypodermic needle (205) has a cross-section configured to accommodate passage and to maintain selected orientation of the at least one retroreflector. Subcutaneous insertion into a selected location (TL) is provided by ejection out of an open-end extremity (217) of a hypodermic needle and by operation in association with an imaging apparatus, which is possibly an ultrasound imaging apparatus.

It is yet a further object of the present invention to provide a method and apparatus wherein subcutaneous insertion into a selected location (TL) is provided by coupling the at least one retroreflector (RR, R1, R2) to at least one surgical thread attached to a surgical needle (205), and pulling the at least one surgical thread trough body tissue (T) until the at least one retroreflector (RR, R1, R2) reaches a selected location and orientation. The at least one surgical thread is (301, 3031) biodegradable surgical thread.

It is yet an additional object of the present invention to provide a method and apparatus wherein the at least one surgical thread is non-biodegradable surgical thread and is appropriately fastened to retain the at least one retroreflector (RR, R1, R2) in the selected position and orientation, and is instrumental for retrieving the at least one retroreflector (RR, R1, R2) out of the body. Moreover, subcutaneous insertion with at least one surgical thread is operated in association with an imaging apparatus, which is possibly an ultrasound imaging apparatus.

It is yet another additional object of the present invention to provide a method and apparatus wherein the apparatus has a sampler (200) configured for emitting radiation and for collecting returned imprinted radiation, a processing unit (300) for processing imprinted radiation and for analysis, and a display (400) for presenting analysis results. Furthermore, the apparatus has a communication unit (500, 501B) configured to transmit data in either one of both and both wireless communication and wire communication.

It is a further object of the present invention to provide a method and apparatus wherein the sampler (100) is configured for emitting radiation in at least one wavelength and for collecting imprinted radiation in same matching at least one wavelength.

One more object of the present invention to provide a method and apparatus wherein the emitted radiation is as selected alone and in combination from the group of radiation consisting of single-wavelength light, multi-wavelength light, a sequence of successive beams of single wavelength light, and successive sets of multi-wavelength light.

One further object of the present invention to provide a method and apparatus wherein the emitted radiation is as selected alone and in combination from the group of radiation consisting of infra-red light, near-infrared light, white light, coherent light, non-coherent light, ultra-violet light.

Moreover, it is an object of the present invention to provide a method and apparatus wherein the analyzer (100) has a display unit (400), which is configured as a touch screen to display analysis results and to receive commands as input to the processing unit (300). Preferably, the analyzer (100) is configured to operate in synchronization with bodily pulsation rhythm.

Furthermore, it is an object of the present invention to provide a method and apparatus wherein the analyzer (100) is configured to operate when worn on the wrist, and the analyte is glucose, or hemoglobin, or a component of interstitial fluid. Likewise, the analyzer (100) is configured to operate when carried in a pocket adjacent the skin (S).

In addition, it is an object of the present invention to provide a method and apparatus wherein the analyzer (100) has an input unit (700), which is configured for entering commands into the processing unit to which it is coupled.

Finally, it is an object of the present invention to provide a method and apparatus wherein the at least one wavelength is selected in association with the blood analyte for which data is derived.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a schematic diagram of the prior art using IR absorption spectroscopy,

FIG. 2 depicts a syringe insertion tool for the subcutaneous insertion by injection of a retroreflector,

FIG. 3 illustrates the insertion of a retroreflector by help of surgical needle and thread,

FIG. 4 is a schematic diagram of a first embodiment 1000 of an apparatus having a sampler operative in association with an implanted retroreflector,

FIG. 5 shows a retroreflector with two levels of retroreflection,

FIG. 6 illustrates a second embodiment 2000 of a sampler device having an optical prism and a diffractive grid,

FIG. 7 shows a source of radiation with a compact multicolor source of collimated illumination,

FIG. 8 is a schematic diagram of a third embodiment 3000 having an implanted retroreflector with two levels of retroreflection and a wideband illumination source,

FIG. 9 shows various possible combinations of retroreflectors.

FIGS. 10, 11 and 12 are block diagrams depicting various implementations,

FIG. 13 illustrates a wrist-worn device, and

FIG. 14 is a block diagram of a wrist-worn device in combination with an implanted retroreflector.

### MODES FOR CARRYING OUT THE INVENTION

The present invention deals mainly with a method and an apparatus for the detection of developments of trends in glucose concentration, and for the measurement of glucose concentration levels, taken in tissues and in blood. However, the method and apparatus described hereinbelow are applicable for deriving biological parameters and for the detection and quantification of other blood and tissues' analytes as well.

For implementation, there is required an initial setup step, consisting of the insertion into a tissue of a minute retroreflector. The retroreflector is possibly implanted in tissue, under a blood vessel, adjacent or in the interior of a muscle, and the like. This minor implanting intervention is judged to be of minimal annoyance to a patient when compared to daily finger pricking.

FIG. 2 shows a cross-section of a syringe insertion tool 201, which is loaded with a retroreflector RR in the form of a small retroreflective pellet 203 contained in the interior of the hypodermic needle 205, ready for injection. The dimensions of the retroreflective pellet 203 are adaptable to match the needs of the application. A typical retroreflective pellet 203 measures about 0.2×0.3×1 mm. However, longer retroreflective elements RR, with the size of 0.5×1×5, or even 0.5×1×6 are also practical.

A piston 207, slidingly engaged into the syringe body 209 of the syringe insertion tool 201, is concentrically coupled to the proximal end 211 of a plunger 213, which is slidingly received in the hypodermic needle 205. The piston 207 protrudes externally outward of the syringe body 209, as is common with conventional hypodermic syringes. However, contrary to conventional hypodermic syringes, the piston 207 allows air to escape when driven into the interior of the body 209.

The distal end 215 of the plunger 213 abuts the retroreflective pellet 203, which is disposed adjacent the open-end extremity 217 of the needle 205. Evidently, the hypodermic needle 205 has a cross-section configured to match and accommodate passage of at least one retroreflective pellet 203. Furthermore, the cross-section of the hypodermic needle 205 is not necessarily circular but is configured, if desired, to match and accommodate, the free passage of a selected cross-section for the at least one retroreflective pellet 203. Such a non-circular cross-section configuration is beneficial to maintain a selected orientation and attitude control of the at least one retroreflector RR, while accommodating passage through the hypodermic needle 205. If desired, more than one retroreflector RR are loaded in sequence in the hypodermic needle 205 for consecutive injection.

A safety-catch 219 terminated by a pull-ring 221 diametrically crosses the syringe body 209 and the piston 207 to lock the piston 207, and the plunger 213 relative to the syringe body 209. Two handles 223 extending radially outward of the body 209 serve as finger restraints. Other safety devices may also be used.

In operation, the syringe insertion tool 201 is taken out of its sterile envelope, not shown in the Figs., and the needle 205 is inserted subcutaneously, via a selected area of the skin, into the tissue. When the open-end extremity 217 of the needle 205 is inserted in the selected location, the pull-ring 221 is pulled, thereby retrieving the safety-catch 219 and liberating the piston 207 in translation, now able to slidingly travel relative to the syringe body 209. A slight push on the protruding extremity of the piston 207 drives the plunger 215, whereby the retroreflective pellet 203 is ejected out of the open-end extremity 217 of the needle 205 and implanted in the tissue in subcutaneous insertion, into the selected location in chosen orientation and attitude. If desired, to ease precise implantation, the insertion process is operated in association with an imaging apparatus, such as ultrasound imaging apparatus.

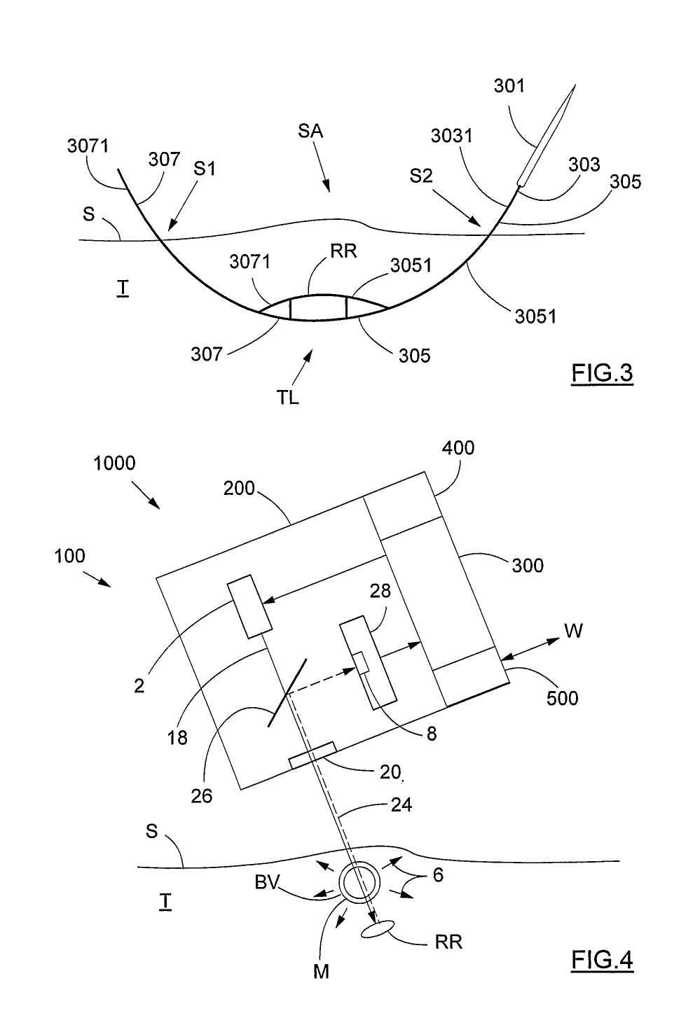

FIG. 3 depicts another retroreflector insertion method. A surgical needle 301 is shown after having pierced the skin S for insertion at an entry location S1 of a selected area SA of the skin S, and after exit thereout through an exit location S2. The surgical needle 301 for subcutaneous insertion is coupled to at least one retroreflector RR, which is attached to at least one surgical thread 303 that is pulled through bodily tissue T until a selected location TL and a chosen orientation are reached. The retroreflector RR is coupled intermediate a leading portion 305 and a trailing portion 307 of the thread 303.

One surgical thread suffices, since a pull on either the leading portion 305 or the trailing portion 307 of the section of the surgical thread 303 that extends above the skin S permits to shift the position of the retroreflector RR toward either one side of the entry location S1 or of the exit location S2. However, a separate second surgical thread 3031, longitudinally contiguous to the first surgical thread 303, allows better control over the orientation of the retroreflector RR before or after reaching the selected location TL.

In FIG. 3, the second surgical thread 3031, which coincides with the first surgical thread 303 over most of its length, is indicated as having a leading portion 3051 and a trailing portion 3071. Evidently, each portion of both the first surgical thread 303 and the second surgical thread 3031 is coupled to a different emplacement of the retroreflector RR to which they are respectively attached, to enhance control of orientation and attitude by appropriate pull on a selected thread portion. Here again, to ease precise implantation, the insertion process is operated, if desired, in association with an imaging apparatus, such as an ultrasound imaging apparatus.

The surgical thread is selected either as biodegradable surgical thread, or as non-degradable surgical thread, which is instrumental for retrieving the at least one retroreflector RR out of the body, if desired. When the at least one surgical thread is non-degradable surgical thread, it is possible to take advantage thereof by appropriate fastening of the surgical thread portions, to ascertain retention of the at least one retroreflector in the selected position and orientation.

A conventional ambulatory micro-surgical procedure is another option for the insertion of a retroreflector RR. A minimal cut in the skin S, possibly not even requiring stitches, is straightforward and suits the requirements.

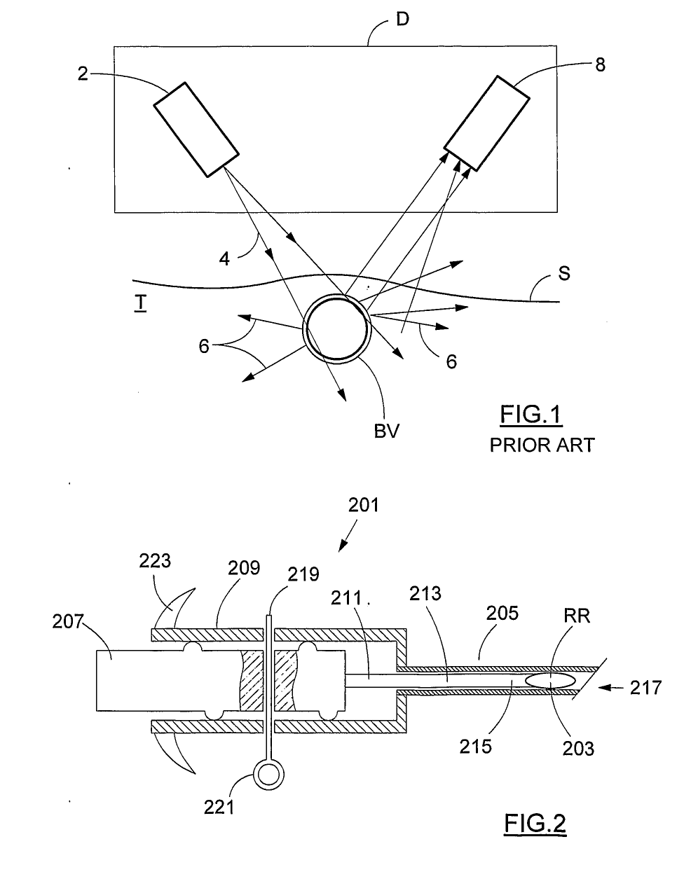

The purpose of using a retroreflective element RR is to increase the sensitivity of the data sampler of the system. First, a retroreflector RR increases the efficiency of illumination, i.e. the proportion of radiation of an illuminating light beam that reaches a sensor pertaining to a detection unit of data sampler. That efficiency increases by orders of magnitude relative to prior art setups without a reflective element, as depicted in FIG. 1.

Second, the retroreflective element RR amplifies the signal-to-noise ratio by minimizing scattered light rays. Thereby, due to the retroreflector RR, the attenuation in the detected wavelengths of radiation is owed to pure light absorption, rather than to the combination of light absorption and light scatter.

Third, the relative contribution of light absorption by tissue or blood is increased. The reflector RR thus serves to improve the sensitivity of the spectral analysis and allows quantification of analytes, such as blood glucose concentrations, with precision levels far better than existing prior art non-invasive devices.

FIG. 4 is a schematic diagram of a first embodiment 1000 of an analyzer 100 with a data sampler 200 having a source of radiation 2, a window 20, a semi-reflective mirror 26, and a detection unit 28 having at least one sensor 8, all operating in association with an implanted retroreflector RR. The source of radiation 2 emits a collimated beam 18 of infrared (IR) light. On its way out of the data sampler 200, the beam 18 passes through the transparent window 20, and then sequentially, through the skin S, tissue T, and a blood vessel BV, to finally reach the retroreflector RR.

In contrast with common optically reflective elements R, retroreflectors RR present the optical property of always reflecting an impinging light beam back in the orientation of the incoming light beam, but in opposite direction toward the source of illumination. Consequently, the use of a retro-reflector RR eliminates constraints imposed on the angle of incoming radiation of illuminating light beams, since the orientation of the reflected light is predetermined and well known. The retroreflector RR thus helps to increase the illumination efficiency of the incoming radiation, as well as the reduction of rays of scattered light 6.

Still in FIG. 4, a portion of the collimated light beam 18 is lost as scattered rays 6, but most of the light beam 18 is reflected back by the retroreflector RR toward the source of radiation 2, as a retroreflected returned beam 24 of imprinted radiation, indicated as a dashed line. The retroreflected returned beam 24 of imprinted radiation enters the data sampler 200 through the transparent window 20, and is deflected by an appropriately oriented semi-reflective mirror 26, toward the detection unit 28. Thanks to the use of a retroreflector RR, the user U, not shown in the Figs., is liberated from the requirement to hold the data sampler 200, or the analyzer 100, in a position that will ensure a rigorously precise orientation of the emitted light beam 18, to ascertain reception of the returned and reflected beam 24 by the detection unit 28. It is assumed that the detection unit 28 has at least one sensor 8. It is noted that the retroreflector RR may be implanted in the interior of a muscle M, or under a muscle M, as shown in FIG. 4.

The detection unit 28 is coupled to a processor unit P for deriving results from the returned imprinted radiation 24 which results are shown on a display 400. The processor unit P also controls the source of radiation 2 and operates and manages the operation of the analyzer 100. Optionally, the processor is coupled to a communication unit 500 for bi-directional communication of data and instructions, as indicated by the double-headed arrow marked W.

Eliminating the constraints on the angle of impinging illumination by use of a retroreflector RR renders the device very simple to use and maximizes the reliability of the results obtained. It is important to emphasize that reliable measurement results are a crucial requirement for self-monitoring blood glucose devices, which are to be operated by patients with varying levels of competence, and yet, must produce accurate and repeatable results. Furthermore, reducing the constraints on the angle of disposition of the data sampler 200 relative to the skin S facilitates continuous monitoring, especially where body movements are expected to affect the alignment of the impinging beam of illumination with the retroreflector RR.

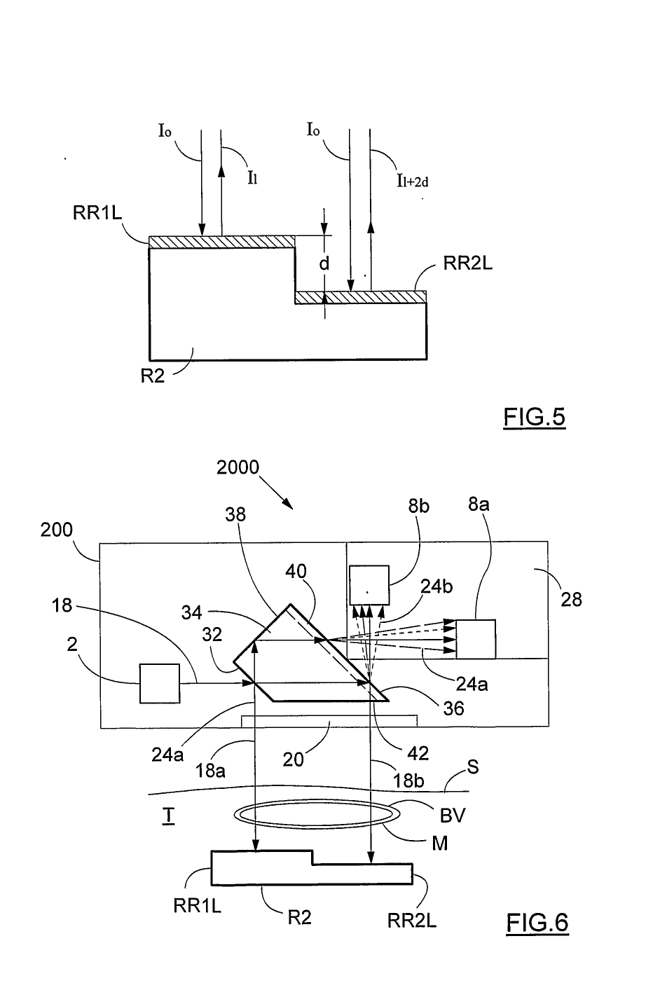

FIG. 5 depicts a retroreflector R2 with two levels of retroreflection for implantation in tissue, featuring a first level of retroreflection RR1L and a second level of retroreflection RR2L. The vertical step distance separating the two levels of retroreflection, thus the height of the step between the proximal retroreflector RR1L and the distal retroreflector RR2L, is indicated as the step distance d.

If desired, the retroreflector RR is chosen with more than two levels of retroreflection. The twin-level retroreflector R2 is further described hereinbelow.

It is well known in the art that for spectrophotometer-based analyzers, the strength of an absorbance/reflectance measurement is proportional to the concentration of the analyte, which is the product of the absorbing substance's molar absorptivity with the substance's concentration, and to the distance, or path length, traveled by the illumination beam, for a given wavelength of light. Thus, according to the absorption coefficient equation, also referred to as the Beer-Lambert Law, it is known that:

$$A = log \frac{{I}_{0}}{{I}_{1}} = a \times l \qquad equ . ( 1 )$$

where:
A is the absorbance,
I₀ is the intensity of the incident light,
I₁ is the intensity of the light after crossing the examined sample,
a is the effective absorption coefficient (product of the absorbing substance's molar absorptivity with the substance's concentration),
1 is the distance traveled by the light beam through the sample.
   Equation equ. (1) is rewritten as

$$a \times l = log \frac{{I}_{0}}{{I}_{1}} \qquad equ . ( 2 )$$

and 1 is taken as the distance traveled by the illumination beam 18, with 1+2d indicating the distance traveled by the same illumination beam 18 down to and up from the distal second level of the retroreflector RR2L. One may write a set of two equations; one for the first distance 1 and one for the second distance 1+2d. Then, by solving the set of two equations, one derives:

$$a = \frac{1}{2 d} \times log \frac{{I}_{0}}{{I}_{l + 2 d}} \qquad equ . ( 3 )$$

However, I₀ and I_{1+2d} are measurable values, and d is the known step distance between the two levels of the twin-level retroreflector R2. In this manner, by use of the twin-level retroreflector R2, the distance 1 traveled by the light beam 18 is factored out from the absorption coefficient equation equ. (1). This means that in practice, since now A=a, the level of absorbance is measured directly by help of I₀, I_{1+2d}, and d. Hence, the use of a two-level retroreflector R2 permits to obtain a reading of the absorbance as a directly measured value.

A further exemplary embodiment for the direct measurement of the level of absorbance in association with a twin-level retro-reflector R2, and with two beams of light is provided hereinbelow, with reference to FIG. 6.

FIG. 6 is a schematic diagram of the optics of a second embodiment 2000, with an optical prism element and a diffraction grating, in addition to the optics of the first embodiment 1000.

In FIG. 6, the source of radiation 2 emits a collimated beam 18 of wideband IR light that impinges on a first slanted semi-reflective surface 32 of an optical prism 34. The semi reflective surface 32 deflects one portion of the impinging beam 18 out of the data sampler 200 as a first deflected beam 18a, via the window 20, skin S and tissue T, toward the proximal first level RR1L of the subcutaneously implanted twin-level retroreflector R2.

The remaining portion of the beam 18 that was not deflected by the semi-reflective surface 32, propagates through the optical prism 34 and is deflected as a second deflected beam 18b, by a second slanted semi-reflective surface 36, out of the prism 34, and out of the data sampler 200, through window 20, skin S, and tissue T, in parallel to the first beam 18a, toward the second distal level RR2L of the twin-level retroreflector R2.

Both first and second parallel beams, respectively 18a and 18b, are reflected by the twin-level retroreflector R2 to return into the data sampler 200 through tissue T, skin S, and window 20, and pass through a diffraction grating 40 before being received by a detection unit 28 having a first sensor 8a, and a second sensor 8b. It is the task of the diffraction grating 40 to spectrally resolve the first and second parallel beams, respectively 18a and 18b, before being received by their respective sensors 8a and 8b.

The first reflected beam 24a retrogrades through the window 20, propagates through the first slanted surface 32 to hit a third slanted surface 38 of the prism 34, from where it is deflected to pass though the second slanted surface 36 and through the diffraction grating 40 disposed thereon, to finally reach the first sensor 8a.

In parallel, the second reflected beam 24b retrogrades through the window 20, propagates in perpendicular to and through the basis 42 of the prism 34, to exit via the diffraction grating 40, and be received by the second sensor 8b.

FIG. 6 thus provides an example of a data sampler 200 for the measurement of the absorbance of a bodily analyte, such as glucose, with a source of radiation 2 having a double beam of illumination and a twin-level retroreflector R2, whereby the optical path length 1 between the radiation source 2 and the retroreflector R2 is eliminated from equation (1) to provide a direct reading of the concentration of glucose, or any other analyte. It is understood that the received radiation 18a and 18b, and returned radiation 24a and 24b, have at least a first portion and a second portion of radiation crossing bodily matter via a separate, respectively, first path, here via the skin S, tissue T, and blood vessel BV, and a second path, which is via the skin S, and the tissue T. The first path and the second path of travel of both portions of radiation thus cross the same bodily matter, as in FIG. 6, or cross different bodily matter, as in FIG. 8 hereinbelow. Other bodily matter is possibly also a muscle M, or any other organ or tissue of the body, although not shown in the Figs.

In FIG. 6, the embodiment 2000 may use a wideband source of radiation 2, emitting light provided by, but not limited to, white LEDs or a cluster of narrowband IR LEDs of different wavelengths, switched on as desired, each one alone, or in selected groups, or all together.

Alternative embodiments may sense the absorption spectrum by using a detection unit 28 with a single wideband sensor, in association with a source of radiation 2 emitting multiple wavelengths. By way of example, diode lasers having a plurality of wavelengths may be used, or IR LEDs of different wavelengths switched on one at a time, or in combination. Either way, finding the effective absorption coefficient at different wavelengths yields the absorption spectrum of the sample under examination, from which the concentration of the analytes, or fluid ingredients can be deduced using standard statistic methods, on the basis of the known absorption spectrum of the analytes.

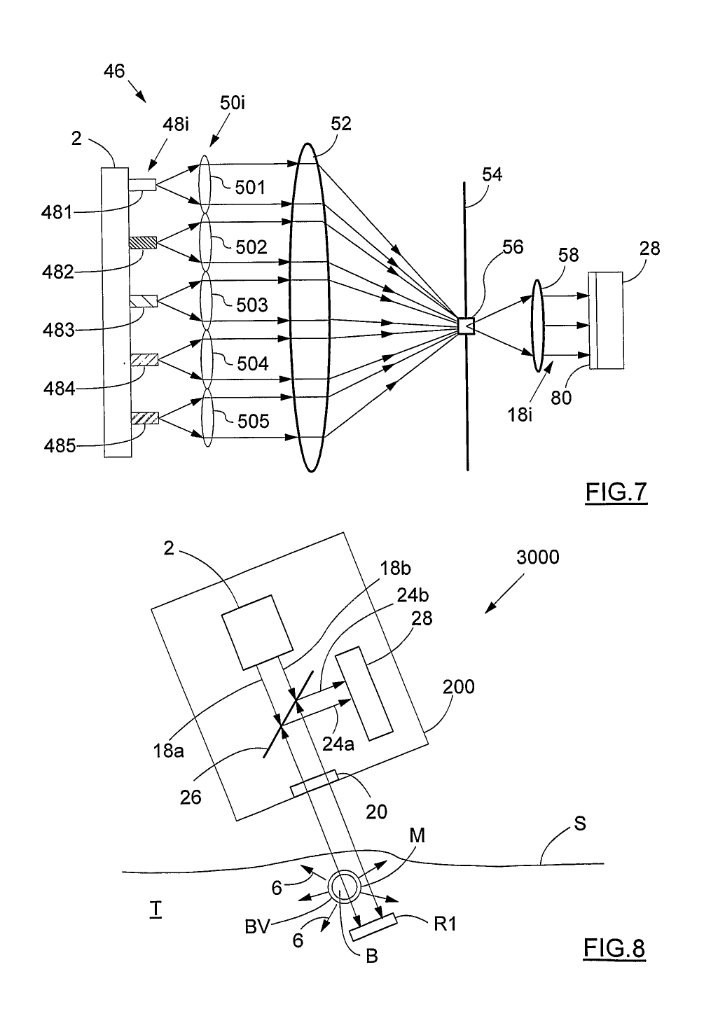

An embodiment implementing a cluster, or an array of narrowband IR light sources, each light source with a different wavelength of light is described hereinafter with reference to FIG. 7.

FIG. 7 shows a source of radiation 2 implemented as a compact multicolor source 46 of collimated illumination, or combiner 46, with a plurality of narrowband IR light sources 48, each light source having a different wavelength and each light source being controllably switched on sequentially, either individually or in a desired combination. For each switched on light source that illuminates the blood vessel BV, a different spectrum response is received by the sensor(s). In FIG. 7 the narrowband IR sources 48 are shown as a linear array of sources 48i, with i=1, 2, . . . , n, but may also be disposed as a two or three-dimensional array of sources 48ijk, with i, j, and k being a series of integers running from 1 to respectively, n, p and q. For the sake of simplicity reference is made to the linear array 48i, although the description applies also to the multi-dimensional arrays of illumination 48ijk. FIG. 7 shows five light sources 48, with i=1, 2, . . . , 5. The light sources i in FIG. 7 are thus numbered consecutively from 481 to 485.

A collimating lens 50, possibly out of a linear array 50i, or out of a two or three-dimensional array of collimating lenses 50ijk is disposed to correspond and in opposite to the IR sources 48 ijk. Reference is made to a linear array of IR sources for the sake of simplicity. Annotation for the collimating lenses 50i conforms to the annotation for the sources 48i. Hence, the five collimating lenses are numbered from 501 to 505, but in FIG. 7, only lenses 501 and 505.

One respective collimator lens 50i, is disposed opposite a corresponding source 48i to collimate the rays emanating from each source 48i onto a collection lens 52, serving to focus these rays onto an iris 54, wherein a diffuser 56 is disposed. Rays from the diffuser 56 impinge on a single collimator 58, to finally exit the compact multicolor source 46 as a narrowband collimated IR beam 18i, having a wavelength according to the selected IR source(s) 48i. The response to the different wavelengths is measured by a single wideband sensor 80, which generates a spectral signature. As an alternative the well-known method of using beam splitting mirrors to combine illumination sources can be used.

A detection unit 28 with a single wideband sensor 80, or receptor 80, is configured to receive the returned retroreflected imprinted radiation, in response to the illumination of the blood vessel BV by different wavelengths 48i, and to derive a spectral signature corresponding to each emitted wavelength.

FIG. 8 is a schematic rendering of a further embodiment 3000 shown with incoming radiation divided into two parallel beams of light. Although not shown in the Figs., the same embodiment is adaptable to operate with more than two beams of light by configuring the source of radiation 2 to emit the desired number i of beams, and by configuring the sensor(s) 8 of the detection unit 28 as either, but not shown in FIG. 8, a single wideband sensor 80, or as a plurality of i narrowband sensors 8i.

FIG. 8 depicts an example of a data sampler 200 utilizing two parallel beams of light to obtain the differential absorption spectrum resulting from a first absorption spectrum derived from the incoming radiation crossing the skin S, tissue T, and blood vessel BV, and from a second absorption spectrum derived from the impinging radiation passing only through the skin S and tissue T. Each beam thus crosses different bodily matter.

The light source 2 emits two identical incoming collimated IR beams, indicated as first beam 18a and second beam 18b. Both incoming beams 181a and 18b pass through a semi-reflective mirror 26, and exit via the transparent window 20, to reach the retroreflector R1 having a single level of retroreflection, whereby they are reflected back along their incoming path, thus by 180°, into the sampler 200 via window 20, as a first reflected beam 24a and as a second reflected beam 24b. Some light from both incoming beams, respectively 18a and 18b, is lost as scattered rays 6, but most of the light form both incoming beams, is reflected by the retroreflector R1 back towards the light source 2.

In FIG. 8 it is shown that the incoming beam 18a and the reflected beam 24a, first reach, and then are returned by the single-level retroreflector R1, after passing through the skin S, tissue T, and the blood vessel BV. In contrast, the incoming beam 18b and the reflected beam 24b reach and are retroreflected by the single-level retroreflector R1 only via the skin S and tissue T, without crossing the blood vessel BV.

Both reflected beams 24a and 24b are deflected by the semi-reflective mirror 26 toward the detection unit 28 that derives the differential absorption spectrum of the blood B on the basis of the absorption spectra measured for both beams 24a and 24b. Taking the difference between the two absorption spectra, namely that of the first returned imprinted beam 24a passing through the blood vessel BV, and that of the second returned imprinted beam 24b laterally bypassing that same blood vessel BV, yields the net absorption spectrum of solely the blood. The differential absorption spectrum is derived by the sampler 200, or in association with, or only by the processing unit 300, not shown in FIG. 8.

The capture of only the net absorption spectrum of the blood B is achieved by taking the difference between the two absorption spectra, namely that of the first beam of returned retroreflected radiation 24a passing through the blood vessel BV, and that of the second beam of returned retroreflected radiation 24b, bypassing that same blood vessel BV. Thereby, the net absorption spectrum of only the blood B is obtained.

Acquiring the net absorption spectrum of the blood B by use of differential measurements eliminates the need for frequent calibration of the data sampler 200. This important feature allows an analyzer 100 incorporating the data sampler 200 as a replacement for, rather than as an addition to devices using standard finger-pricking methods and devices. An analyzer 100 with a data sampler 200 is thus able to monitor absolute glucose levels as well as glucose level trends. The same holds true for other analytes too.

Various embodiments of apparatus configured to emit and collect radiation in association with a retroreflector RR have been described hereinabove. Radiation is emitted as at least one beam having at least one wavelength, or with a plurality of wavelengths, or as successive beams of different wavelength. In matching correspondence with the emitted radiation, returned radiation is collected by a detection unit 28 with at least one sensor 8 for one wavelength, or with a plurality of sensors where each sensor 8i is dedicated to one wavelength, or with a single wideband sensor 80.

Multiple configurations of implanted retroreflectors RR are possibly selected to suit various needs, in association with data samplers and computer application programs running on the processing unit 300 for deriving results from the readings of the data sampler.

Radiation is directed subcutaneously, toward a blood vessel BV, or another organ, or tissue T. Each combination provides practical results for deriving the net concentration of blood analytes such as glucose, as described hereinbelow.

It is possible to select many different configurations for the optics of the data sampler 200. This is achieved by selecting alone and in combination, from practical options regarding the number of emitted beams of radiation, the wavelength of the radiation, and the impingement disposition relative to the chosen type or retroreflective element RR. In general, additional rays of illumination benefit the reliability of the readings of the data sampler 200.

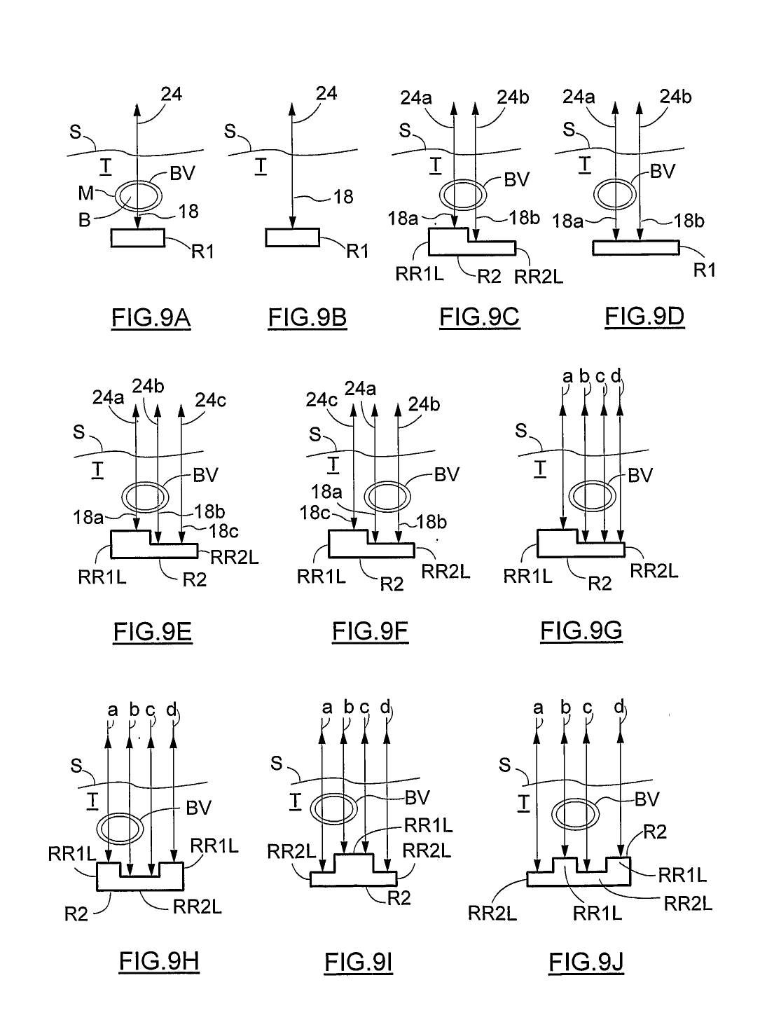

FIGS. 9A to 9J illustrates exemplary options available.

FIG. 9A depicts the embodiment 1000 showing one beam of radiation 18 crossing the skin S, tissue T, and the blood vessel BV. The incoming radiation 18 is reflected as a retroreflected returned beam 24 of imprinted radiation by the retroreflector R1 having a single level of retroreflection. The target is the blood vessel BV, to derive data from the blood. As described hereinabove, embodiment 1000 permits to derive the concentration of blood analytes, such as glucose.

Another method of obtaining the glucose concentration of the blood B alone would be to synchronize spectral measurements with blood pulsation. If desired, spectral measurements taken by use of the embodiment 1000 are enhanced by synchronization of the data sampling in association with the rhythm of pulsation of the blood B. Since the blood volume in a blood vessel is higher during the high-pressure portion of the pulse rhythm than it is in the low-pressure phase, the difference between the absorption spectra obtained in the high-pressure portion of the pulse rhythm and the low-pressure phase thereof is the net result of absorption by the blood B alone.

For further enhancement, beams of radiation of different wavelength also improve detection accuracy.

If desired for calibration purposes, a single measurement is taken by conventional means from a sample of blood, for example by finger pricking. Once calibrated for a given user U, not shown in the Figs., net concentration results are derived. Alternatively, the readings derived by the data sampler 200 are calibrated by using standard statistical methods.

Calibration by use of a blood sample taken in vivo or by statistical methods, synchronization with blood pulsation, and illumination with beams of light of various wavelengths are techniques applicable to the different cases described, and are referred to as enhancement techniques.

It is noted that in the same manner, as illustrated in FIG. 9A, a muscle M, or another organ, may replace the blood vessel BV for the derivation of analyte data therefrom. A blood vessel BV is illustrated in FIGS. 9A to 9J, but is provided as an example only and may be replaced by a muscle M, or by another organ, or may be deleted from the Figs., to illustrate data derivation only from tissue T.

FIG. 9B is the same as FIG. 9A, but without the blood vessel BV. The target is thus the interstitial fluid in the tissue T, from which glucose concentration data is derived for example.

FIG. 9C relates to the embodiment 2000 shown in FIG. 6 and described hereinabove, where the radiation is separated into two impinging beams of light, reflected from a retroreflector R2 having two levels of retroreflection. When the blood vessel BV is removed from the drawing of Fig. C, but not shown in a separate drawing, then another exemplary option is provided for the derivation of data from the interstitial fluid in the tissue T.

FIG. 9D relates to the embodiment 3000 shown in FIG. 8. Enhancement techniques described hereinabove are evidently applicable to embodiments emitting two and more beams of light.

FIGS. 9E and 9E relate to radiation with three impinging beams of light in association with an implanted retroreflector R2 having two levels of reflection.

In FIG. 9E, one incoming beam of radiation, or light beam 18a crosses skin S, tissue T, and blood vessel BV, to reach the proximal first level of retroreflection RR1L, and be returned as a retroreflected beam 24a of imprinted radiation. The second parallel incoming beam of radiation 18b also crosses skin S, tissue T, and blood vessel BV, but reaches the distal second level of retroreflection RR2L, and is returned as a retroreflected beam 24b of imprinted radiation. These two beams thus represent the embodiment 2000, depicted in FIG. 9C. An additional parallel third beam of incoming radiation 18c crosses only the skin S and tissue T, before reaching the distal second level of retroreflection RR2L, to be returned as retroreflected beam 24c of imprinted radiation. This third beam of radiation is a reference beam permitting to derive results by differentiation and combinations of various kinds, to be performed by the processing unit 300.

For FIG. 9F, the sole difference relative to FIG. 9E is that the incoming beam 18c, which is returned as imprinted beam 24c, reaches the proximal first level of retroreflection RR1L, and not the second level of retroreflection RR2L.

Three beams of radiation associated with a retroreflector RR having two levels of retroreflection provide better measurement accuracy and reliability for analyte concentration level derivation, or for glucose level concentration or trends measurements.

FIGS. 9G to 9J are four Figs. relating to radiation with four impinging beams of light associated with an implanted retroreflector R2 having two levels of retroreflection. For the sake of clarity, each one of the four impinging and returned beams is designated only by the previously used suffix, thus successively as a, b, c, and d.

In all four FIGS. 9G to 9J, one couple of beams cross the blood vessel BV as described relative to the embodiment 2000, and the second couple of light beams are reference beams, each one of both beams being received and returned interchangeably by a first proximal level and a second distal level of retroreflection. FIG. 9G depicts the basic conceptual implementation, while FIGS. 9H to 9J present variations in the implementation of the twin-level retroreflector R2.

In the FIGS. 9G, 9H, 9I, and 9J, the couple of light beams crossing the blood vessel BV, alike implementation 2000 of FIG. 9C, are designated as, respectively, b and c, a and b, a and b, and b and c. For the same FIGS. 9G, 9H, 9I, and 9J, the couple of reference beams are designated as, respectively, a and d, c and d, c and d, and finally, a and d.

A mirror-view of FIG. 9J is also possible but is not shown in the Figs. Evidently retroreflectors with more than two levels of retroreflection are also offers implementation possibilities, but are not shown in the Figs. for the sake of repetitiveness.

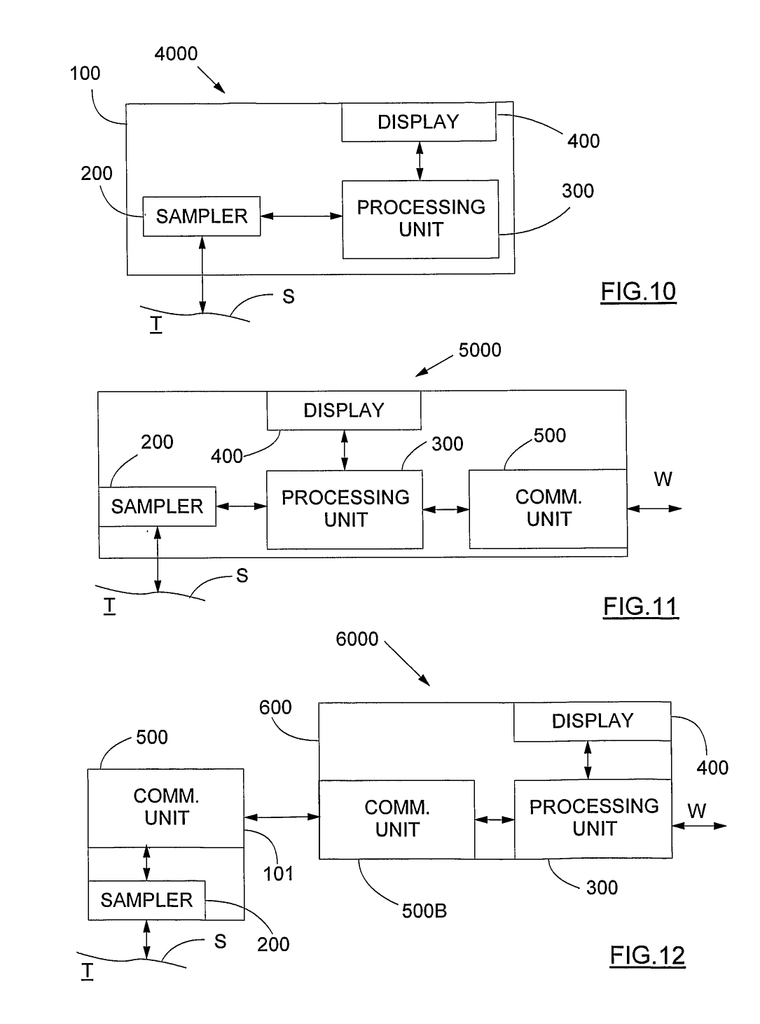

FIG. 10 shows an embodiment 4000 of an analyzer 100 with a sampler 200. The sampler 200 is optically coupled to the skin S and also coupled in bi-directional communication with the processing unit 300 and with the display 400 whereupon results are presented. If desired, the display 400 is configured as an I/O device, such as a touch screen, or a separate I/O device is coupled to the processing unit 300, although not shown in FIG. 10, for the sake of simplicity. Input commands are entered via the display 400, or separate I/O device, either to the processing unit 300, or to the sampler 200 via the processing unit 300.

As described hereinabove, the sampler 200 takes analyte readings under command of the processing unit 300. These analyte readings are forwarded to the processing unit 300 for the derivation of data to be shown as results on the display 400. The processing unit 300 is configured appropriately to read and run application computer programs, in association with a memory for the storage of those application computer programs and of instructions, as well as for the storage of data, present and historical.

All the analyzers 100 are powered by a power supply, such as a battery, possibly rechargeable, but not shown in the Figs. for the sake of clarity.

FIG. 11 illustrates an embodiment 5000 similar to the embodiment 4000 shown in FIG. 10, but with the addition of a data communication unit 500 that is coupled in bi-directional communication with the processing unit 300 and with the sampler 200. The processing unit is thus in bi-directional communication with the sampler 200, the display 400, and the communication unit 500. Thereby, the analyzer 100 and the sampler 200, are able to emit and receive data and instructions, either in wireless communication or by wire, as shown by the doubled-headed arrow W.

FIG. 12 depicts an embodiment 6000 having a two-part, or split analyzer 101 operating in association with a support unit 600. The split analyzer 101 has a sampler 200 coupled to a first communication unit 500 for the transmission of collected data to the support unit 600. The support unit 600 carries a second communication unit 500B, which is coupled in bi-directional communication with the first communication unit 500. A processing unit 300 is coupled to the second communication unit 500B and to a display 400.

The split analyzer 101 collects data in the same manner as the sampler 100, but transmits the collected data to the support unit 600 for further processing and display of results as described hereinabove.

In practice, the analyzer 100, or 101 is carried adjacent the skin S of a user U who is not shown, or disposed in a garment, such as in a pocket of shirt, close to the skin S and to the retroreflector RR.

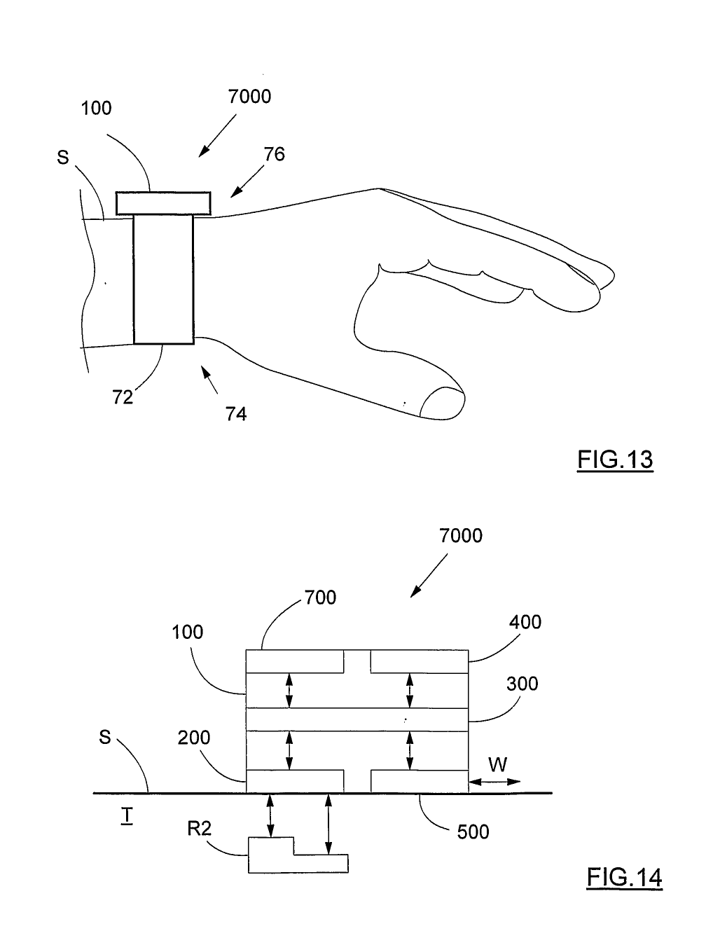

FIG. 13 illustrates an example of an embodiment 7000 of an analyzer 100 retained by a wristband 72 to the wrist 74 of a user U, which is not shown in the Figs. It is noted that the analyzer 100 may be retained either in continuation of the back of the hand 76 as shown in FIG. 13, or be rotated by half a circle to be worn in continuation of the palm of the hand, depending on the location of the implanted retroreflector RR.

FIG. 14 schematically depicts some elements of the embodiment 7000 of the wrist-worn analyzer 100. Under command and control of the processing unit 300, the data sampler 200 obtains retroreflected returned beams of imprinted radiation from the twin-level retroreflector R2, which returned beams are processed and the results are forwarded, to the display 400. If the display 400 is configured to accept input commands, these may be entered as instructions to the processing unit 300, which is coupled in bi-directional communication with the display 400. In the alternative, a separate input unit 700 is disposed in the analyzer 100, for providing instructions to processing unit 300. Instructions to the data sampler 200 are transmitted by bi-directional communication via the processing unit 300. If desired, a communication unit 500 coupled to the processing unit 300 is configured to emit and receive data and commands, as indicated by the double-headed arrow W.

For operation, the analyzer 100 is worn by the user U, and if desired, is calibrated for the first-time use. Calibration is achieved by any of the known methods, for example, by comparison with the results provided by a standard blood-pricking device. Calibration data is entered into the analyzer 100 by help of the touch screen display 400, or via the communication unit 500, or via the dedicated input device 700.

### INDUSTRIAL APPLICABILITY

Industrial applicability is self-evident and similar to that of other analyzers used for the benefit of users.

It will be appreciated by persons skilled in the art, that the present invention is not limited to what has been particularly shown and described hereinabove. Rather, the scope of the present invention is defined by the appended claims and includes both combinations and subcombinations of the various features described hereinabove as well as variations and modifications thereof which would occur to persons skilled in the art upon reading the foregoing description.

1-80. (canceled)

81. A method for monitoring bodily analytes by use of an analyzer (100) configured for emitting radiation oriented to impinge on a selected area (SA) of the skin (S), for collecting returned imprinted radiation exiting the skin, for processing and analysis of the imprinted radiation, and for display of analysis results,
the method being characterized by the steps of:
inserting at least one retroreflector (RR, R1, R2) subcutaneously to the selected area (SA) for receiving radiation and returning retroreflected radiation, and
collecting retroreflected radiation exiting from the selected area as returned imprinted radiation.

82. The method according to claim 81, wherein:
imprinted radiation is collected separately from each at least one retroreflector (RR, R1, R2).

83. The method according to claim 81, wherein: the at least one retroreflector (RR, R1, R2) has at least one level of retroreflection (RR1L, RR2L).

84. The method according to claim 81, wherein:
the at least one retroreflector (RR, R1, R2) is disposed appropriately and configured to return retroreflected radiation as a plurality of distinct separate imprinted beams collected for either one of both separate and combined processing and analysis.

85. The method according to claim 81, wherein:
the at least one retroreflector (RR, R1, R2) is configured and disposed in selected position and orientation to receive radiation and return radiation crossing at least the skin (S) and tissue (T).

86. The method according to claim 81, wherein:
the received and returned radiation have at least a first portion and a second portion of radiation crossing bodily matter via a separate, respectively, first path and a second path, and
the first path and the second path cross different bodily matter.

87. The method according to claim 81, wherein: subcutaneous insertion into a selected location (TL) is provided by ejection of the at least one retroreflector (RR, R1, R2) out of a hypodermic needle (205).

88. The method according to claim 87, wherein:
subcutaneous insertion into a selected location (TL) is provided by ejection out of an open-end extremity (217) of a hypodermic needle and by operation in association with an imaging apparatus.

89. The method according to claim 88, wherein:
the imaging apparatus is an ultrasound imaging apparatus.

90. The method according to claim 81, wherein:
subcutaneous insertion into a selected location (TL) is provided by coupling the at least one retroreflector (RR, R1, R2) to at least one surgical thread attached to a surgical needle (205), and pulling the at least one surgical thread trough body tissue (T) until the at least one retroreflector (RR, R1, R2) reaches a selected location and orientation.

91. The method according to claim 81, wherein:
the apparatus has a sampler (200) configured for emitting radiation and for collecting returned imprinted radiation, a processing unit (300) for processing imprinted radiation and for analysis, and a display (400) for presenting analysis results.

92. The method according to claim 91, wherein:
the sampler (100) is configured for emitting radiation in at least one wavelength and for collecting imprinted radiation in same matching at least one wavelength.

93. The method according to claim 91, wherein:
emitted radiation is as selected alone and in combination from the group of radiation consisting of single-wavelength light, multi-wavelength light, a sequence of successive beams of single wavelength light, and successive sets of multi-wavelength light.

94. The method according to claim 91, wherein:
emitted radiation is as selected alone and in combination from the group of radiation consisting of infra-red light, near-infrared light, white light, coherent light, non-coherent light, ultra-violet light.

95. The method according to claim 81, wherein:
the analyte is glucose.

96. The method according to claim 81, wherein:
the analyte is hemoglobin.

97. The method according to claim 81, wherein:
the analyte is a component of interstitial fluid.

98. An apparatus for monitoring bodily analytes by use of an analyzer (100) configured for emitting radiation oriented to impinge on a selected area (SA) of the skin (S), for collecting returned imprinted radiation exiting the skin, for processing and analysis of the imprinted radiation, and for display of analysis results,
the apparatus being characterized by:
at least one retroreflector (RR, R1, R2) being inserted subcutaneously to the selected area (SA) for receiving radiation and returning retroreflected radiation, and
retroreflected radiation exiting from the selected area being collected as returned imprinted radiation.

99. The apparatus according to claim 98, wherein:
imprinted radiation is collected separately from each at least one retroreflector (RR, R1, R2).

100. The apparatus according to claim 98, wherein:
the at least one retroreflector (RR, R1, R2) is disposed appropriately and configured to return retroreflected radiation as a plurality of distinct separate imprinted beams collected for either one of both separate and combined processing and analysis.

101. The apparatus according to claim 98, wherein:
the at least one retroreflector (RR, R1, R2) is configured and disposed in selected position and orientation to receive radiation and return radiation crossing at least the skin (S) and tissue (T).

102. The apparatus according to claim 98, wherein:
the apparatus has a sampler (200) configured for emitting radiation and for collecting returned imprinted radiation, a processing unit (300) for processing imprinted radiation and for analysis, and a display (400) for presenting analysis results.

103. The apparatus according to claim 102, wherein:
the apparatus has a communication unit (500, 501B) configured to transmit data in either one of both and both wireless communication and wire communication.

104. The apparatus according to claim 102, wherein:
the sampler (100) is configured for emitting radiation in at least one wavelength and for collecting imprinted radiation in same matching at least one wavelength.

105. The apparatus according to claim 102, wherein:
emitted radiation is as selected alone and in combination from the group of radiation consisting of single-wavelength light, multi-wavelength light, a sequence of successive beams of single wavelength light, and successive sets of multi-wavelength light.

106. The apparatus according to claim 102, wherein:
emitted radiation is as selected alone and in combination from the group of radiation consisting of infra-red light, near-infrared light, white light, coherent light, non-coherent light, ultra-violet light.

107. The apparatus according to claim 102, wherein:
the analyzer (100) has a display unit (400) which is configured as a touch screen to display analysis results and to receive commands as input to the processing unit (300).

108. The apparatus according to claim 98, wherein:
the analyte is glucose.

109. The apparatus according to claim 98, wherein:
the analyzer (100) has an input unit (700) which is configured for entering commands into the processing unit to which it is coupled.

110. The apparatus according to claim 98, wherein:
the analyte is hemoglobin.

111. The method according to claim 98, wherein:
the analyte is a component of interstitial fluid.
